(19) United States
(12) Patent Application Publication
(10) Pub. No.: US 2026/0345520 A1
(43) Pub. Date: Nov. 5, 2026

(54) AN ORALLY DISINTEGRATING TABLET OF SERTRALINE AND ITS PROCESS OF PREPARATION

(71) Applicant: NOVUMGEN LIMITED, London (GB)

(72) Inventor: Kamleshkumar Patel, London (GB)

(21) Appl. No.: 19/487,171

(22) PCT Filed: May 28, 2024
(86) PCT No.: PCT/EP2024/064656

§ 371 Date: Nov. 24, 2025

(30) Foreign Application Priority Data
May 29, 2023 (EP) 2308008.8

(51) Int. Cl. A61K 31/135; A61K 9/00; A61K 9/20

(52) U.S. Cl. CPC A61K 31/135; A61K 9/0056; A61K 9/2013; A61K 9/2018; A61K 9/2054; A61K 9/2059; A61K 9/2095

(57) ABSTRACT

The present invention relates to an orally disintegrating tablet comprising sertraline or pharmaceutically acceptable salts thereof, at least one disintegrant, at least one diluent, at least one binder, at least one lubricant, and one or more pharmaceutically acceptable excipients. The orally disintegrating tablet prepared using these excipients exhibits desirable properties such as facilitating disintegration, and dissolution of the drug for oral administration. The present invention also relates to the process for preparing the said solid pharmaceutical composition.

### FIELD OF THE INVENTION

The present invention relates to a pharmaceutical composition of sertraline. The present invention relates to an orally disintegrating tablet of sertraline or pharmaceutically acceptable salts thereof for oral administration. The present invention also relates to the process of the preparation of the same.

### BACKGROUND OF THE INVENTION

Sertraline was first disclosed in the U.S. Pat. No. 4,536,518. Sertraline hydrochloride belongs to a class of antidepressant agents known as selective serotonin-reuptake inhibitors (SSRIs). SSRIs effectively inhibit the reuptake of neuronal serotonin, but they have minimal impact on the reuptake of norepinephrine or dopamine and do not antagonize α- or β-adrenergic, dopamine D2, or histamine H1 receptors. During acute use, SSRIs prevent serotonin reuptake and increase the serotonin stimulation of somatodendritic 5-HT1A and terminal autoreceptors, while chronic use causes desensitization of somatodendritic 5-HT1A and terminal autoreceptors. The resulting increase in serotonergic neurotransmission leads to adaptive changes in neuronal function.

Sertraline selectively inhibits the reuptake of serotonin (5-HT) at the presynaptic neuronal membrane, thereby increasing serotonergic activity. This results in an increased synaptic concentration of serotonin in the CNS, which leads to various functional changes that are associated with enhanced serotonergic neurotransmission. These changes are believed to be responsible for the antidepressant action and beneficial effects in obsessive-compulsive and other anxiety-related disorders.

Sertraline is indicated for the management of major depressive disorder (MDD), post-traumatic stress disorder (PTSD), obsessive-compulsive disorder (OCD) in adults and pediatric patients 6-17 years and older, panic disorder (PD), premenstrual dysphoric disorder (PMDD), and social anxiety disorder (SAD).

The IUPAC name of sertraline is (1S,4S)-4-(3,4-dichlorophenyl)-N-methyl-1,2,3,4-tetrahydronaphthalen-1-amine and the chemical structure is as follows

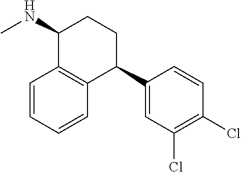 [SMILES: CN[C@H]1CC[C@@H](c2ccc(Cl)c(Cl)c2)c2ccccc21]

Sertraline hydrochloride, which is classified as a BCS class II drug due to its high permeability and low solubility, has an elimination plasma half-life of approximately 26 hours. The solubility and particle size of the drug are closely related, as reducing the particle size of the drug substance can increase its surface area, leading to improved solubility and dissolution rate. In the present invention, tablets prepared with sertraline exhibiting a range of particle sizes have been found to possess the desired disintegration properties, indicating that this tablet formulation could facilitate drug dissolution and absorption.

Sertraline is available as tablets, capsules, oral solutions, oral concentrate, and oral suspension. The marketed products of sertraline in tablet form are available in three dosage strengths: 25 mg, 50 mg, and 100 mg, while capsules are available in two dosage strengths: 150 mg and 200 mg dosages. The oral concentrate is available in 20 mg/ml. The oral suspension is available in 50 mg/5 ml and the oral solution is available in two strengths: 20 mg/ml, and 100 mg/5 ml.

There are many prior arts disclosing different formulations of sertraline as described below:

U.S. Pat. No. 4,962,128 discloses an oral tablet and capsule of sertraline. The tablet is made by blending sertraline hydrochloride, sodium citrate, alginic acid, polyvinylpyrrolidone, and magnesium stearate together in specific weight proportions, and then tablets are punched from the resulting mixture. The capsule is made by combining sertraline hydrochloride, calcium carbonate, and polyethylene glycol in specific weight proportions, and then thoroughly agitating the dried mixture to obtain a uniform powdered product. Finally, the powdered product is used to fill soft elastic and hard-filled gelatin capsules.

U.S. Pat. No. 6,727,283 discloses an essentially non-aqueous, filterable, liquid concentrate solution containing sertraline hydrochloride, ethanol, glycerine, and one or more pharmaceutically acceptable excipient. The process involves dissolving butylhydroxytoluene (BHT), in a portion of the ethanol, which is then added to the main compounding tank containing the glycerin and the remainder of the ethanol. Menthol and Sertraline hydrochloride is then Sequentially added and mixed to dissolve. The sertraline hydrochloride is intentionally added to the solution last to optimize the protective effect of the antioxidant (BHT). The compounded solution is passed through a filter, and the final product is filled into appropriate containers, such as an amber glass bottle with a child-resistant closure.

EP0980241 B1 discloses a gelatin-encapsulated composition comprising sertraline, or a pharmaceutically acceptable salt thereof, and a water-immiscible vehicle. The process involves dissolving the sertraline base or its salts, in an appropriate water-immiscible vehicle, and encapsulating the solution in a soft or hard gelatin capsule by conventional methodology.

US 2002/0132854A1 discloses an intramuscular injection containing sertraline in microparticles that release the drug over an extended period of time. The process involves preparing an aqueous phase (Solution A) and an organic phase (Solution B), which are then mixed together with a sertraline base to form microparticles. These microparticles are washed, dried, and then suspended in an aqueous vehicle before being frozen and lyophilized. The lyophilized microparticles can be sterilized with gamma irradiation and reconstituted with sterile water prior to injection. This injection allows for effective treatment without multiple dosing, as the sertraline is released slowly over time after administration.

The solid dosage form of sertraline presently available has relatively long onset times, which can cause delayed therapeutic effects. Moreover, it also presents a challenge for patients who may have difficulty swallowing tablets and capsules, leading to poor patient compliance. In contrast, the liquid dosage form is bulky, difficult to transport, and takes up a lot of space. Due to their inherent instability, liquid dosage forms often have shorter shelf lives. The patient's measurement of the exact volume determines whether the dose is administered appropriately, which increases the chance for variability. Therefore, alternative dosage forms that are easier to swallow and have a faster onset time need to be explored. An orally disintegrating tablet of the present invention is a pharmaceutical formulation specifically designed in such a way that the entire tablet disintegrates with saliva within a short time when put in the mouth, facilitating easy administration to patients. The orally disintegrating tablet of sertraline is the best-suited dosage form to formulate, as it offers several benefits, including precise dosing, fast disintegration in the oral cavity without the need for water, fast dissolution, and improved patient adherence, especially for patients who have difficulty swallowing, such as paediatrics and geriatrics suffering from dysphagia. The present invention solves all prior art problems and provides a pharmaceutical composition for oral administration comprising sertraline.

### SUMMARY OF THE INVENTION

In accordance with the present invention, the orally disintegrating tablet for oral administration is prepared. The orally disintegrating tablet comprises sertraline or pharmaceutically acceptable salts thereof, at least one disintegrant, at least one diluent, at least one binder, at least one lubricant, and one or more pharmaceutically acceptable excipients.

Another embodiment relates to a pharmaceutical orally disintegrating tablet which consists essentially of 0.05% w/w to about 15% w/w, preferably in the range from about 0.1% w/w to about 10% w/w sodium starch glycolate, wherein the tablet exhibits disintegration within less than 3 minutes, preferably less than 2 minutes.

Another embodiment of the present invention is to provide a process for obtaining said orally disintegrating tablet via the wet granulation method.

Another embodiment of the present invention is to provide taste-masking properties and present pleasant palatability such that the administration of the orally disintegrating tablet is not unpleasant for children, the elderly, or unconscious patients, and thus patient compliance is improved.

Further another embodiment of the present invention is directed to a method of preparing compositions comprising: (a) Sieving sertraline or pharmaceutically acceptable salts thereof, diluent, and disintegrant are sieved separately through a 40 #sieve, and sweetener, flavoring agent, and lubricant separately through a 60 #sieve; (b) Mixing of previously sifted sertraline hydrochloride, and diluent into the rapid mixer granulator and mix for 10 minutes by keeping the impeller slow and the chopper off; (c) Preparing binder solution by adding in 75% of purified water with continuous stirring until it becomes a clear solution; (d) Adding binder solution slowly to the dry mix with continuous mixing in a rapid mixer granulator for 10 min by keeping the impeller slow and the chopper off and then granulating the mixture for a further 2 min with impeller slow and chopper on with intermittent addition of a remaining quantity of purified water; (e) Drying the above-granulated blend in a fluidized bed dryer at 50 °C±5 °C till the loss on drying of the dried granules becomes not more than 1.0-2.0%; (f) passing the dried granules through a 24 #sieve; (g) Mixing previously sifted disintegrant, sweetener, and flavoring agent to the dried granules prepared in step (f) and blending for 10 minutes in a blender; (h) Adding lubricant to the pre-lubricated blend of step (g) and mix properly for 5 minutes; and (i) Compressing the resulting mixture into the tablet dosage form.

Another embodiment of the present invention can effectively treat major depressive disorder (MDD), post-traumatic stress disorder (PTSD), obsessive-compulsive disorder (OCD) in adults and paediatric patients 6-17 years and older, panic disorder (PD), premenstrual dysphoric disorder (PMDD), and social anxiety disorder (SAD).

### OBJECTS OF THE INVENTION

The primary object of the present invention is to provide an orally disintegrating tablet of sertraline or pharmaceutically acceptable salts thereof.

Another object of the present invention is to provide a therapeutically effective amount of sertraline or pharmaceutically acceptable salts thereof, sodium starch glycolate, mannitol, microcrystalline cellulose, magnesium stearate, hydroxypropyl cellulose, and one or more pharmaceutically acceptable excipients.

Another object of the present invention is to enhance the disintegration and dissolution of the drug for oral administration.

Another object of the present invention is to improve the solubility of sertraline or pharmaceutically acceptable salts which belong to BCS Class-II drugs with poor solubility.

Another further object of the present invention is to provide an orally disintegrating tablet, which is particularly convenient for administration for pediatric and geriatric patients.

A further object of the present invention is to provide a stable orally disintegrating tablet of sertraline or pharmaceutically acceptable salts thereof.

Yet another object of the present invention is to provide a treatment that is effective and very convenient for administration without the need for water or swallowing difficulties, which will improve patient compliance.

### DETAILED DESCRIPTION OF THE INVENTION

The present invention is understood more readily by reading the following detailed description of the invention and by studying the included examples.

The term “Orally disintegrating tablet” refers to a solid dosage form of the present invention, which disintegrates rapidly in the oral cavity of a patient after administration, without chewing.

The term “about” is used herein to mean approximately, in the region of, roughly, or around. When the term “about” is used in conjunction with a numerical range, it modifies that range by extending the boundaries above and below the numerical values set forth. In general, the term “about”, as and when used in this specification, means±10% of the mentioned value.

The term “% w/w” is intended to mean the percentage of an ingredient(s)/the total percentage by weight of the composition (100%). Here the % w/w is to be calculated against the total weight of the composition.

The main embodiment of the present invention is an orally disintegrating tablet comprised of sertraline or pharmaceutically acceptable salts thereof, at least one disintegrant and diluent. In addition, the orally disintegrating tablet further comprises at least one pharmaceutically acceptable excipient selected from a binder, sweetener, flavoring agent, and lubricant.

As per one embodiment of the present invention sertraline or pharmaceutically acceptable salts thereof, is present in the range of about 20% w/w to about 60% w/w, preferably in the range of about 25% w/w to about 45% w/w. The D90 particle size of sertraline or a pharmaceutically acceptable salt thereof is less than 150 μm, more preferably less than 100 μm. The D50 particle size of sertraline or a pharmaceutically acceptable salt thereof is less than 50 μm, more preferably less than 20 μm. The D10 particle size of sertraline or a pharmaceutically acceptable salt is less than 20 μm, more preferably less than 10 μm.

As per one embodiment, a suitable diluent for the present invention is selected from the group consisting of cellulose derivative, microcrystalline cellulose, microfine cellulose, sugar alcohols such as maltitol, xylitol, mannitol, sorbitol, erythritol, dextrates, dextrose, natural polymers, lactose, starch, pregelatinized starch, and dextrin, or any combinations thereof. In the present invention, the combination of cellulose derivative and sugar alcohol is preferred as a diluent in the range of about 30% w/w to about 80% w/w, preferably in the range from about 40% w/w to about 60% w/w. Microcrystalline cellulose is present in the range from about 25% w/w to about 65% w/w, preferably from about 30% w/w to about 50% w/w, and mannitol is present in the range from about 0.04% w/w to about 25% w/w, preferably from about 0.5% w/w to about 15% w/w. The use of mannitol as a filler in tablet development has increased due to its various physicochemical properties, including high flowability, non-hygroscopicity, chemical inertness, and advantageous compactibility during tableting. By incorporating mannitol into the tablet formulation, both the mechanical properties of the tablet and the flow of the powder can be improved, ensuring uniform compression. Mannitol is a crystalline material with excellent compressibility, capable of forming a strong bond with other tablet components. This feature helps to prevent tablet defects by ensuring that the tablets are uniformly compressed and have adequate binding between their components. Furthermore, granulations containing mannitol are easy to dry. Apart from these benefits, mannitol's negative heat of solution, sweetness, and mouthfeel not only enhances the perception of flavors but also helps with optimizing taste-masked formulations. On the other hand, microcrystalline cellulose is a type of filler that facilitates efficient dry blending of ingredients and enables excellent tablet compression. Its swelling tendencies and exceptional water imbibing or wicking action make it an ideal filler for the wet granulation process. Moreover, its wicking action encourages rapid wetting of the powder mixture, reduces sensitivity to over-wetting, and accelerates the drying process, resulting in faster tablet disintegration. Thus, the combination of microcrystalline cellulose and mannitol is preferred over other diluents in the present invention.

As per one another embodiment, a suitable disintegrant for the present invention is selected from a group consisting of methylcellulose, alginic acid, guar gum, carboxymethylcellulose calcium, polacrilin potassium, croscarmellose sodium, carmellose calcium, crospovidone, calcium hydrogen phosphate, anhydrous calcium hydrogen phosphate, sodium carboxymethyl starch, poloxamer, magnesium aluminum silicate, sodium starch glycolate, and sodium alginate or any combination thereof. Sodium Starch glycolate is the preferred disintegrant for the present invention in the range from about 0.05% w/w to about 15% w/w, preferably in the range from about 0.1% w/w to about 10% w/w. Sodium starch glycolate has excellent hydration capacity, flow properties, and rapid water absorption, which causes significant swelling and a notable increase in the volume of granules. As a result, the tablets disintegrate rapidly and uniformly. Therefore, sodium starch glycolate is an ideal disintegrant for orally disintegrating tablets of sertraline Hydrochloride.

As per one more embodiment of the present invention, a suitable lubricant is selected from the group consisting of boric acid, magnesium stearate, sodium stearyl fumarate, micronized polyoxy ethylene glycol, leucine, sodium benzoate, sodium acetate, sodium lauryl sulfate, stearic acid, sodium stearate, sodium oleate, calcium stearate, waxes or any combination thereof. Magnesium stearate is preferred as a lubricant for the present invention and is present in the range from about 0.05% w/w to about 25% w/w, preferably in the range from about 0.2% w/w to about 15% w/w, wherein the ratio of magnesium stearate to sertraline hydrochloride is 1:5 to 1:35 preferably in the range of about 1:10 to 1:30.

As per one embodiment, a suitable binder for the present invention can be selected from the group consisting of alginic acid, carbomer, ethyl cellulose, gelatine, glucose, gum acacia, sodium alginate, guar gum, hydroxyethyl cellulose, hypromellose, polyvinylalcohol, carboxyvinyl polymers, carboxymethyl ethyl cellulose, hydroxypropyl cellulose, hydroxypropyl methylcellulose, methylcellulose, polydextrose, polyethylene oxide, methacrylate copolymers, ethyl cellulose, aminoalkyl methacrylate copolymers, polyvinylpyrrolidone or any combination thereof. Hydroxypropyl cellulose is preferred as a binder for the present invention. It is present in the range from about 0.025% w/w to about 20% w/w, preferably in the range from about 0.05% w/w to about 10% w/w.

As per another embodiment of the present invention, at least one further pharmaceutically acceptable excipient is a sweetener selected from the group consisting of cyclamate, acesulfame potassium, neo hesperidin dihydrochalcone, monoammonium glycyrrhizinate, saccharin sodium, sucralose, saccharin, aspartame or any combination thereof. Sucralose is preferred as a sweetener for the present invention and is present in the range of about 0.05% w/w to about 25% w/w, preferably in the range of about 0.2% w/w to about 15% w/w.

As per one another embodiment of the present invention, a flavoring agent is selected from a group consisting of menthol, floral fennel flavor, mint powder, vanillin, orange flavor, or any combinations thereof. The orange flavor is preferred as a flavoring agent present in the range from about 0.05% w/w to about 20% w/w, preferably in the range from about 0.2% w/w to about 15% w/w.

In the preferred embodiment, the present invention has been made to solve the above-mentioned problems, and its object is to use sertraline or a pharmaceutically acceptable salt thereof as an active ingredient, which provides a faster disintegration time and faster dissolution rate. Sertraline is used for the treatment or prevention of major depressive disorder (MDD), post-traumatic stress disorder (PTSD), obsessive-compulsive disorder (OCD) in adults and pediatric patients 6-17 years and older, panic disorder (PD), premenstrual dysphoric disorder (PMDD), and social anxiety disorder (SAD) and very convenient for administration without the problem of swallowing and using water thereby increasing patient compliance.

As per one embodiment of the present invention, the orally disintegrating tablet comprising selective serotonin-reuptake inhibitors (SSRIs) or pharmaceutically acceptable salts thereof, present in an amount from about 20% w/w to about 60% w/w, preferably in the range of about 25% w/w to about 45% w/w, a diluent is present in the range of about 30% w/w to about 80% w/w, preferably in the range from about 40% w/w to about 60% w/w, a disintegrant is present in the range from about 0.05% w/w to about 15% w/w, preferably in the range from about 0.1% w/w to about 10% w/w, lubricant is present in the range from about 0.05% w/w to about 25% w/w, preferably in the range from about 0.2% w/w to about 15% w/w, a binder is present in the range of about 0.025% w/w to 20% w/w, preferably in the range of about 0.05% w/w to about 10% w/w, the sweetener is present in the range of about 0.05% w/w to about 25% w/w, preferably in the range of about 0.2% w/w to about 15% w/w and the flavoring agent is present in the range from about 0.05% w/w to about 20% w/w, preferably in the range from about 0.2% w/w to about 15% w/w.

As per one preferred embodiment of the present invention comprising sertraline or pharmaceutically acceptable salts thereof, present in an amount from about 20% w/w to about 60% w/w, preferably in the range of about 25% w/w to about 45% w/w, the combination of cellulose derivative and sugar alcohol is present in the range of about 30% w/w to about 80% w/w, preferably in the range from about 40% w/w to about 60% w/w, microcrystalline cellulose is present in the range from about 25% w/w to about 65% w/w, preferably from about 30% w/w to about 50% w/w, mannitol is present in the range from about 0.04% w/w to about 25% w/w, preferably from about 0.5% w/w to about 15% w/w, sodium starch glycolate is present in the range from about 0.05% w/w to about 15% w/w, preferably in the range from about 0.1% w/w to about 10% w/w, magnesium stearate is present in the range from about 0.05% w/w to about 25% w/w, preferably in the range from about 0.2% w/w to about 15% w/w, hydroxypropyl cellulose is present in the range of about 0.025% w/w to 20% w/w, preferably in the range of about 0.05% w/w to about 10% w/w, sucralose is present in the range of about 0.05% w/w to about 25% w/w, preferably in the range of about 0.2% w/w to about 15% w/w and orange flavour is present in the range from about 0.05% w/w to about 20% w/w, preferably in the range from about 0.2% w/w to about 15% w/w.

As per one more embodiment of the present invention, the ratio of lubricant to the drug is in the range of 1:5 to 1:35 preferably in the range of about 1:10 to 1:30. The ratio of magnesium stearate to sertraline hydrochloride is in the range of 1:5 to 1:35 preferably in the range of about 1:10 to 1:30.

Another embodiment of the present invention is to manufacture an orally disintegrating tablet containing sertraline by wet granulation method, which is one of the most economical methods.

As per one another embodiment, the orally disintegrating tablet is devoid of any inorganic diluent.

As per one another embodiment, the disintegrating time of the sertraline orally disintegrating tablet is less than 3 minutes, preferably less than 2 minutes.

As per another embodiment, 90% of the sertraline is released in 45 minutes, preferably more than 95% is released within 30 minutes.

As per one embodiment of the present invention, packaging material for an orally disintegrating tablet of sertraline is selected from the Polypropylene Bottle with Silica canister, PP Bottle with an Oxygen scavenger, HDPE Bottle, HDPE Bottle with SAF, HDPE Bottle with CR cap, HDPE Bottle with Silica canister, HDPE Bottle with an Oxygen scavenger, Alu-Alu Blister and PVC/PVDC-Alu Film. In the present invention, the PVC/PVDC-Alu Film or HDPE Bottle with SAF closure is preferred as packaging material to deliver the desired physicochemical parameters.

As per one more embodiment, the wet granulation method is used to prepare the orally disintegrating tablet. sertraline hydrochloride, mannitol, microcrystalline cellulose, and sodium starch glycolate are sieved separately through a 40 #sieve. Sucralose, orange flavor, and magnesium stearate are sieved separately through a 60 #sieve. The previously sifted mannitol, microcrystalline cellulose, and sertraline hydrochloride were loaded into the rapid mixer granulator and mixed for 10 minutes by keeping the impeller slow and the chopper off. The binder solution was prepared by dissolving hydroxypropyl cellulose in 75% of purified water with continuous stirring until it becomes a clear solution, then slowly adding it to the dry mix with continuous mixing in a rapid mixer granulator for 10 min by keeping the impeller slow and the chopper off and then granulating the mixture for a further 2 min with impeller slow and chopper on with intermittent addition of a remaining quantity of purified water. The granulated blend is then dried in a fluidized bed dryer at 50 °C±5 °C till the loss on drying of the dried granules becomes not more than 1.0-2.0%. The dried granules were sifted through a 24 #sieve. To the dried granules, the previously sifted sodium starch glycolate, sucralose, and the orange flavor were added and blended for 10 minutes in a blender and then magnesium stearate was added to the pre-lubricated blend and mixed for 5 minutes. Finally, the lubricated mixture is compressed to form tablets. The prepared tablets were packed in PVC/PVDC-Alu Film or HDPE Bottle with SAF closure preferred as packaging materials to deliver the desired physicochemical parameters.

The invention is further illustrated by the following examples, which are by no means intended to limit the scope of the invention but are given by way of illustration.

### Example 1

The orally disintegrating tablet was made according to the method defined below using the formulation having the ingredients shown in Table I for different dose strengths are 20 mg, 50 mg, and 100 mg of sertraline:

TABLE-I

| Ingredients | % w/w |
|---|---|
| Sertraline hydrochloride | 37.33 |
| Microcrystalline Cellulose | 43.00 |
| Mannitol | 6.67 |
| Sodium starch glycolate | 1.33 |
| Hydroxypropyl cellulose | 3.33 |
| Sucralose | 2.67 |
| Orange flavor | 2.67 |
| Magnesium Stearate | 3.00 |
| Total | 100.00 |

### Manufacturing Process:

Sertraline Hydrochloride, mannitol, microcrystalline cellulose, sodium starch glycolate, and hydroxypropyl cellulose are sieved separately through a 40 #sieve. Sucralose, orange flavor, and magnesium stearate are sieved separately through a 60 #sieve. In a blender, mannitol, microcrystalline cellulose, and sertraline hydrochloride are blended and mixed for 15 min. The previously sifted sodium starch glycolate, hydroxypropyl cellulose, sucralose, and the orange flavor were added to the above blend and mixed for 10 min. At last, the previously sifted magnesium stearate is added to the above mixture and blended for 5 min.

### Observation:

The blend flow was not good so, further compression activity was not performed.

To optimize blend flow, it was necessary to change the method from dry granulation to wet granulation.

| Test Parameters of blend | Result |
|---|---|
| Bulk Density (gm/ml) | 0.292 |
| Tap Density (gm/ml) | 0.403 |
| Carr Index (%) | 27.5 |
| Hausner Ratio | 1.37 |

### Example 2

The orally disintegrating tablet was made according to the method defined below using the formulation having the ingredients shown in Table II for different dose strengths are 20 mg, 50 mg, and 100 mg of sertraline:

TABLE-II

| Ingredients | % w/w |
|---|---|
| Sertraline hydrochloride | 37.33 |
| Microcrystalline Cellulose | 43.00 |
| Mannitol | 6.67 |
| Sodium starch glycolate | 1.33 |
| Hydroxypropyl cellulose | 3.33 |
| Purified water | Q. S |
| Sucralose | 2.67 |
| Orange flavor | 2.67 |
| Magnesium Stearate | 3.00 |
| Total | 100.00 |

### Manufacturing Process:

Sertraline Hydrochloride, mannitol, microcrystalline cellulose, and sodium starch glycolate are sieved separately through a 40 #sieve. Sucralose, orange flavor, and magnesium stearate are sieved separately through a 60 #sieve. The previously sifted mannitol, microcrystalline cellulose, and Sertraline Hydrochloride were loaded into the rapid mixer granulator and mixed for 10 minutes by keeping the impeller slow and the chopper off. The binder solution was prepared by dissolving hydroxypropyl cellulose in 75% of purified water with continuous stirring until it becomes a clear solution, then slowly adding it to the dry mix with continuous mixing in a rapid mixer granulator for 10 min by keeping the impeller slow and the chopper off and then granulating the mixture for a further 2 min with impeller slow and chopper on with intermittent addition of a remaining quantity of purified water. The Granulated blend is then dried in a fluidized bed dryer at 50 °C±5 °C till the loss on drying of the dried granules becomes not more than 1.0-2.0%. The dried granules were sifted through a 24 #sieve. To the dried granules, the previously sifted sodium starch glycolate, sucralose, and the orange flavor were added and blended for 10 minutes in a blender and then magnesium stearate was added to the pre-lubricated blend and mixed for 5 minutes. Finally, the lubricated mixture is compressed to form tablets.

### Observation:

The blend flow was proper but a Capping problem was observed during compression.

To minimize the Capping problem, it was necessary to decrease the concentration of Lubricant.

| Test Parameters of blend | Result |
|---|---|
| Bulk Density (gm/ml) | 0.400 |
| Tap Density (gm/ml) | 0.520 |
| Carr Index (%) | 23.08 |
| Hausner Ratio | 1.30 |

The tablets present the characteristics mentioned in the table below:

| Test Parameters | Specification | Result |
|---|---|---|
| Hardness | 10N to 50N | 24N |
| Average weight | 150.0 mg ± 7.5% | 147.35 mg |
| | (138.75 mg to 161.25 mg) | |

### Example 3

The orally disintegrating tablet was made according to the method defined below using the formulation having the ingredients shown in Table III for different dose strengths are 20 mg, 50 mg, and 100 mg of sertraline:

TABLE-III

| Ingredients | % w/w |
|---|---|
| Sertraline hydrochloride | 37.33 |
| Microcrystalline Cellulose | 44.00 |
| Mannitol | 6.67 |
| Sodium starch glycolate | 1.33 |
| Hydroxypropyl cellulose | 3.33 |
| Purified water | Q. S |
| Sucralose | 2.67 |
| Orange flavor | 2.67 |
| Magnesium Stearate | 2.00 |
| Total | 100.00 |

### Manufacturing Process:

Sertraline Hydrochloride, mannitol, microcrystalline cellulose, and sodium starch glycolate are sieved separately through a 40 #sieve. Sucralose, orange flavor, and magnesium stearate are sieved separately through a 60 #sieve. The previously sifted mannitol, microcrystalline cellulose, and Sertraline Hydrochloride were loaded into the rapid mixer granulator and mixed for 10 minutes by keeping the impeller slow and the chopper off. The binder solution was prepared by dissolving hydroxypropyl cellulose in 75% of purified water with continuous stirring until it becomes a clear solution, then slowly adding it to the dry mix with continuous mixing in a rapid mixer granulator for 10 min by keeping the impeller slow and the chopper off and then granulating the mixture for a further 2 min with impeller slow and chopper on with intermittent addition of a remaining quantity of purified water. The Granulated blend is then dried in a fluidized bed dryer at 50 °C±5 °C till the loss on drying of the dried granules becomes not more than 1.0-2.0%. The dried granules were sifted through a 24 #sieve. To the dried granules, the previously sifted sodium starch glycolate, sucralose, and the orange flavor were added and blended for 10 minutes in a blender and then magnesium stearate was added to the pre-lubricated blend and mixed for 5 minutes. Finally, the lubricated mixture is compressed to form tablets.

### Observation:

Disintegration time was more than 3 minutes observed during the compression.

In order to minimize disintegration time, it was necessary to decrease the concentration of the binder.

| Test Parameters of blend | Result |
|---|---|
| Bulk Density (gm/ml) | 0.400 |
| Tap Density (gm/ml) | 0.510 |
| Carr Index (%) | 21.60 |
| Hausner Ratio | 1.28 |

The tablets present the characteristics mentioned in the table below:

| Test Parameters | Specification | Result |
|---|---|---|
| Hardness | 10N to 50N | 28N |
| Average weight | 150.0 mg ± 7.5% | 152.20 mg |
| | (138.75 mg to 161.25 mg) | |
| Disintegration | NMT 3 min | 4 min 10 sec |

### Example 4

The orally disintegrating tablet was made according to the method defined below using the formulation having the ingredients shown in Table IV for different dose strengths are 20 mg, 50 mg, and 100 mg of sertraline:

TABLE-IV

| Ingredients | % w/w |
|---|---|
| Sertraline hydrochloride | 37.33 |
| Microcrystalline Cellulose | 46.67 |
| Mannitol | 6.67 |
| Sodium starch glycolate | 1.33 |
| Hydroxypropyl cellulose | 0.67 |
| Purified water | Q. S |
| Sucralose | 2.67 |
| Orange flavor | 2.67 |
| Magnesium Stearate | 2.00 |
| Total | 100.00 |

### Manufacturing Process:

Sertraline Hydrochloride, mannitol, microcrystalline cellulose, and sodium starch glycolate are sieved separately through a 40 #sieve. Sucralose, orange flavor, and magnesium stearate are sieved separately through a 60 #sieve. The previously sifted mannitol, microcrystalline cellulose, and sertraline Hydrochloride were loaded into the rapid mixer granulator and mixed for 10 minutes by keeping the impeller slow and the chopper off. The binder solution was prepared by dissolving hydroxypropyl cellulose in 75% of purified water with continuous stirring until it becomes a clear solution, then slowly adding it to the dry mix with continuous mixing in a rapid mixer granulator for 10 min by keeping the impeller slow and the chopper off and then granulating the mixture for a further 2 min with impeller slow and chopper on with intermittent addition of a remaining quantity of purified water. The Granulated blend is then dried in a fluidized bed dryer at 50 °C±5 °C till the loss on drying of the dried granules becomes not more than 1.0-2.0%. The dried granules were sifted through a 24 #sieve. To the dried granules, the previously sifted sodium starch glycolate, sucralose, and the orange flavor were added and blended for 10 minutes in a blender and then magnesium stearate was added to the pre-lubricated blend and mixed for 5 minutes. Finally, the lubricated mixture is compressed to form tablets. The prepared tablets were packed in PVC/PVDC-Alu Film or HDPE Bottle with SAF closure preferred as packaging materials to deliver the desired physicochemical parameters.

### Observation:

All the physical and chemical parameters of the tablets were found satisfactory.

| Test Parameters of blend | Result |
|---|---|
| Bulk Density (gm/ml) | 0.385 |
| Tap Density (gm/ml) | 0.488 |
| Carr Index (%) | 21.15 |
| Hausner Ratio | 1.27 |

### Example 5

### The Dissolution Profile of the Tablet Prepared According to Example 4

The conditions of dissolution are the following:
Apparatus: USP type II (paddle)
Rate of rotation: 75 RPM
Volume: 900 ml
Temperature: 37 °C±0.5 °C.
Detection: High-performance liquid chromatography equipped with UV/PDA at 265 nm.
Dissolution medium: 4.5 pH acetate buffer

The orally disintegrating tablet of sertraline was tested for its dissolution profile measured in 900 mL of 4.5 pH acetate buffer at 75 RPM in USP II (Paddle) apparatus and the active ingredient of the tablet released more than 95% in 30 minutes.

### Example 6

The tablet prepared according to example 4 were subjected to a stability study of 25 °C/60% RH and 40 °C/75% RH for 3 and 6 months. Results are tabulated below.

### Sertraline 50 mg Orally Disintegrating Tablet

| | | | | Exp 4 (After 3 months) | | | | Exp 4 (After 6 months) | | | |
|---|---|---|---|---|---|---|---|---|---|---|---|
| | | Exp 4 | | 25 °C/ | | 40 °C/ | | 25 °C/ | | 40 °C/ | |
| Test | Specification | (Initial) | | 60% RH | | 75% RH | | 60% RH | | 75% RH | |
| Hardness | 10N to 50N | 26N | | 21N | | 27N | | 24N | | 22N | |
| Average weight | 150.0 mg ± 7.5% (138.75 | 152.10 | mg | 151.24 | mg | 150.58 | mg | 150.67 | mg | 148.44 | mg |
| | mg to 161.25 mg) | | | | | | | | | | |
| Disintegration | NMT 3 min | 31 | sec | 32 | sec | 35 | sec | 42 | sec | 43 | sec |

| Assay (%) | 95.0%-105.0% | 101.7% | 99.4% | 99.5% | 98.9% | 100.3% |
|---|---|---|---|---|---|---|
| Dissolution | NLT 75% (Q) labeled | 95.4 | 96.8 | 97.5 | 99.0 | 98.6 |
| | amount of sertraline | | | | | |
| | should be dissolved | | | | | |
| | in 30 minutes. | | | | | |

Impurities (%)

| Impurity C | Not more than 0.8% | 0.009 | 0.008 | 0.009 | 0.006 | 0.007 |
|---|---|---|---|---|---|---|
| R-Mandelic Acid | Not more than 0.4% | ND | ND | ND | ND | ND |
| Single maximum unknown | Not more than 0.2% | 0.008 | 0.007 | 0.007 | 0.008 | 0.008 |
| impurity | | | | | | |
| Total unknown impurities | Not more than 1.5% | 0.008 | 0.007 | 0.007 | 0.008 | 0.008 |
| including (R)-Mandelic acid | | | | | | |
| Total Impurity | Not more than 2.0% | 0.017 | 0.015 | 0.016 | 0.015 | 0.015 |

1. An orally disintegrating tablet of sertraline for oral administration comprising sertraline or pharmaceutically acceptable salts thereof, is present in an amount ranging from about 20% w/w to about 60% w/w, preferably in the range from about 25% w/w to about 45% w/w;
at least one diluent;
at least one disintegrant;
at least one lubricant;
at least one binder; and one and more pharmaceutical acceptable excipients
wherein the D90 particle size of sertraline or a pharmaceutically acceptable salt thereof is less than 150 μm, more preferably less than 100 μm, the D50 particle size of sertraline or a pharmaceutically acceptable salt thereof, is less than 50 μm, more preferably less than 20 μm, D10 particle size of sertraline or a pharmaceutically acceptable salt thereof, is less than 20 μm, more preferably less than 10 μm;
the orally disintegrating tablet is devoid of any inorganic diluent.

2. The orally disintegrating tablet according to claim 1, wherein the diluent is selected from cellulose derivatives, sugar alcohols, natural polymers, and dextrin or any combinations thereof.

3. The orally disintegrating tablet according to claim 2, wherein the diluent is a combination of cellulose derivative and sugar alcohol present in the range of about 30% w/w to about 80% w/w, preferably in the range of about 40% w/w to about 60% w/w.

4. The orally disintegrating tablet according to claim 3, wherein the sugar alcohol is mannitol present in the range from about 0.04% w/w to about 25% w/w, preferably from about 0.5% w/w to about 15% w/w.

5. The orally disintegrating tablet according to claim 3, wherein the cellulose derivate is microcrystalline cellulose present in the range from about 25% w/w to about 65% w/w, preferably from about 30% w/w to about 50% w/w.

6. The orally disintegrating tablet according to claim 1, wherein the disintegrant is selected from the group consisting of methylcellulose, alginic acid, carboxymethylcellulose calcium, guar gum, polacrilin potassium, croscarmellose sodium, carmellose calcium, crospovidone, calcium hydrogen phosphate, anhydrous calcium hydrogen phosphate, sodium carboxymethyl starch, poloxamer, magnesium aluminum silicate, sodium starch glycolate, and sodium alginate or any combination thereof.

7. The orally disintegrating tablet according to claim 6, wherein the disintegrant is sodium starch glycolate present in the range of about 0.05% w/w to about 15% w/w, preferably in the range of about 0.1% w/w to about 10% w/w.

8. The orally disintegrating tablet according to claim 1, wherein the lubricant is selected from the group consisting of boric acid, magnesium stearate, sodium stearyl fumarate, micronized polyoxy ethylene glycol, leucine, sodium benzoate, sodium acetate, sodium lauryl sulfate, stearic acid, sodium stearate, sodium oleate, calcium stearate, waxes or any combination thereof.

9. The orally disintegrating tablet according to claim 8, wherein the lubricant is magnesium stearate present in the range of about 0.05% w/w to about 25% w/w, preferably in the range from about 0.2% w/w to about 15% w/w.

10. The orally disintegrating tablet according to claim 1, wherein the ratio of a lubricant to the drug is in the range of 1:5 to 1:35 preferably it is in the range of about 1:10 to 1:30.

11. The orally disintegrating tablet according to claim 10, wherein the ratio of magnesium stearate to sertraline hydrochloride is in the range of 1:5 to 1:35 and preferably in the range of 1:10 to 1:30.

12. The orally disintegrating tablet according to claim 1, wherein the binder is selected from the group consisting of alginic acid, carbomer, ethyl cellulose, gelatine, glucose, guar gum, hydroxy ethyl cellulose, hydroxypropyl cellulose, hydroxypropyl methylcellulose, methylcellulose, polydextrose, polyethylene oxide, polyvinylpyrrolidone or any combination thereof.

13. The orally disintegrating tablet according to claim 12, wherein the binder is hydroxypropyl cellulose present in the range from about 0.025% w/w to about 20% w/w, preferably in the range from about 0.05% w/w to about 10% w/w.

14. The orally disintegrating tablet according to claim 1, wherein the sweetener is selected from the group consisting of cyclamate, acesulfame potassium, neo hesperidin dihydrochalcone, monoammonium glycyrrhizinate, sucralose, saccharin, aspartame or any combinations thereof.

15. The orally disintegrating tablet according to claim 1, wherein the flavoring agent is selected from the group consisting of menthol, floral fennel flavor, mint powder, vanillin, orange flavor, or any combinations thereof.

16. The orally disintegrating tablet according to claim 1, further comprises sucralose present in the range of about 0.05% w/w to about 25% w/w, preferably in the range of about 0.2% w/w to about 15% w/w and orange flavor present in the range from about 0.05% w/w to about 20% w/w, preferably in the range from about 0.2% w/w to about 15% w/w.

17. The orally disintegrating tablet according to claim 1, wherein the orally disintegrating tablet is manufactured by the wet granulation method comprising the steps of:
(a) Sieving sertraline or pharmaceutically acceptable salts thereof, microcrystalline cellulose, mannitol, and sodium starch glycolate are sieved separately through a 40 #sieve, and sucralose, orange flavor, and magnesium stearate separately through a 60 #sieve;
(b) Mixing of previously sifted sertraline hydrochloride, mannitol, and microcrystalline cellulose into the rapid mixer granulator and mix for 10 minutes by keeping the impeller slow and the chopper off;
(c) Preparing binder solution by adding hydroxy propyl cellulose in 75% of purified water with continuous stirring until it becomes a clear solution;
(d) Adding binder solution slowly to the dry mix with continuous mixing in a rapid mixer granulator for 10 min by keeping the impeller slow and the chopper off and then granulating the mixture for a further 2 min with impeller slow and chopper on with intermittent addition of a remaining quantity of purified water;
(e) Drying the above-granulated blend in a dryer at 50 °C±5 °C till the loss on drying of the dried granules becomes not more than 1.0-2.0%;
(f) Passing the dried granules through a 24 #sieve;
(g) Mixing previously sifted sodium starch glycolate, sucralose, and orange flavor to the dried granules prepared in step (f) and blending for 10 minutes in a blender;
(h) Adding magnesium stearate to the pre-lubricated blend of step (g) and mix properly for 5 minutes; and
(i) Compressing the resulting mixture into the tablet dosage form.

18. The orally disintegrating tablet according to claim 1, wherein the orally disintegrating tablet is disintegrated upon contact with saliva in less than 3 minutes, preferably less than 2 minutes.

19. The orally disintegrating tablet according to claim 1, is used for the treatment of major depressive disorder (MDD), post-traumatic stress disorder (PTSD), obsessive-compulsive disorder (OCD) in adults and pediatric patients 6-17 years and older, panic disorder (PD), premenstrual dysphoric disorder (PMDD), and social anxiety disorder (SAD).

20. The orally disintegrating tablet according to claim 1, wherein:
a) 20% w/w to about 60% w/w, preferably in the range of about 25% w/w to about 45% w/w of sertraline or pharmaceutically acceptable salts thereof;
b) a combination of cellulose derivative and sugar alcohol present in the range of about 30% w/w to about 80% w/w, preferably in the range of about 40% w/w to about 60% w/w;
c) 25% w/w to about 65% w/w, preferably from about 30% w/w to about 50% w/w of microcrystalline cellulose;
d) 0.04% w/w to about 25% w/w, preferably from about 0.5% w/w to about 15% w/w of mannitol;
e) 0.05% w/w to about 15% w/w, preferably in the range of about 0.1% w/w to about 10% w/w of sodium starch glycolate;
f) 0.05% w/w to about 25% w/w, preferably in the range from about 0.2% w/w to about 15% w/w of magnesium stearate;
g) 0.025% w/w to about 20% w/w, preferably in the range from about 0.05% w/w to about 10% w/w of hydroxy propyl cellulose;
h) 0.05% w/w to about 25% w/w, preferably in the range of about 0.2% w/w to about 15% w/w of sucralose; and
i) 0.05% w/w to about 20% w/w, preferably in the range from about 0.2% w/w to about 15% w/w of orange flavor.
